(19) United States
(12) Patent Application Publication
(10) Pub. No.: US 2026/0066054 A1
(43) Pub. Date: Mar. 5, 2026

(54) DEVELOPER COMPOSITION FOR METAL-CONTAINING PHOTORESIST, AND METHOD OF FORMING PATTERNS INCLUDING DEVELOPING STEP USING THE COMPOSITION

(71) Applicant: SAMSUNG SDI CO., LTD., Yongin-si (KR)

(72) Inventors: Taeksoo KWAK, Suwon-si (KR); Hyungrang MOON, Suwon-si (KR); Jongpil HO, Suwon-si (KR); Seung HAN, Suwon-si (KR); Taeho KIM, Suwon-si (KR); Gyeonghun PARK, Suwon-si (KR); Rosa SHIN, Suwon-si (KR); Eunyoung LIM, Suwon-si (KR); Jamin KU, Suwon-si (KR)

(21) Appl. No.: 19/316,798

(22) Filed: Sep. 2, 2025

(30) Foreign Application Priority Data
Sep. 5, 2024 (KR) 10-2024-0121008

(51) Int. Cl. G16C 10/00; G03F 7/004; G03F 7/16

(52) U.S. Cl. CPC G16C 10/00; G03F 7/0042; G03F 7/168

(57) ABSTRACT

A developer composition for a metal-containing photoresist and a method of forming patterns utilizing the developer composition are disclosed. The developer composition may include a metal compound, an organic solvent, and an additive and may be applied to a metal-containing photoresist having an exposed portion and an unexposed portion. In the unexposed portion, the number of hydrogen bonds between the metal compound and the organic solvent relative to the number of hydrogen bonds between the metal compound and the additive in the metal-containing photoresist may be greater than 0 and less than or equal to about 10, and the number of hydrogen bonds between the additive and the organic solvent relative to the number of hydrogen bonds between the metal compound and the additive in the metal-containing photoresist may be greater than 0 and less than or equal to about 5.

### CROSS-REFERENCE TO RELATED APPLICATION

The present application claims priority to and the benefit of Korean Patent Application No. 10-2024-0121008, filed on Sep. 5, 2024, in the Korean Intellectual Property Office, the entire content of which is incorporated herein by reference.

### BACKGROUND

### 1. Field

One or more embodiments of the present disclosure relate to a developer composition for a metal-containing photoresist and a method of forming patterns that includes a developing step (e.g., act or task) utilizing the developer composition.

### 2. Description of the Related Art

The semiconductor industry has experienced a continuous reduction in critical dimensions, necessitating the development of high-performance photoresist materials and patterning methods capable of supporting increasingly smaller features.

Related art chemically amplified (CA) photoresists are designed for high sensitivity. However, their typical elemental composition—often including oxygen (O), fluorine (F), sulfur(S), and carbon (C)—results in low absorbance at a wavelength of approximately (about) 13.5 nm, thereby reducing sensitivity under extreme ultraviolet (EUV) exposure. Additionally, CA photoresists tend to exhibit increased line edge roughness (LER) at smaller feature sizes, particularly due to the nature of acid-catalyzed processes, where LER increases as photospeed decreases. These limitations highlight the need for new types (kinds) of high-performance photoresists.

Accordingly, there is a need or desire for photoresist materials that offer improved etching resistance and resolution, while concurrently (simultaneously) enhancing sensitivity, critical dimension (CD) uniformity, and LER characteristics in photolithography processes.

### SUMMARY

One or more aspects of embodiments of the present disclosure are directed toward a developer composition for a metal-containing photoresist.

One or more aspects of embodiments of the present disclosure are directed toward a method of forming patterns including a developing step (e.g., act or task) utilizing the developer composition for a metal-containing photoresist.

Additional aspects of embodiments will be set forth in part in the description which follows and, in part, will be apparent from the description or may be learned by practice of the presented embodiments of the disclosure.

According to one or more embodiments, a developer composition for a metal-containing photoresist is provided. The developer composition is applied to a metal-containing photoresist having exposed and unexposed portions, and includes:

a metal compound; an organic solvent; and an additive.

In the unexposed portion of the photoresist, the number of hydrogen bonds between the metal compound and the organic solvent, relative to the number of hydrogen bonds between the metal compound and the additive in the metal-containing photoresist, is greater than 0 and less than or equal to about 10.

Additionally, the number of hydrogen bonds between the additive and the organic solvent, relative to the number of hydrogen bonds between the metal compound and the additive in the metal-containing photoresist, is greater than 0 and less than or equal to about 5.

The number of hydrogen bonds is determined by averaging the count per frame from a molecular dynamics simulation utilizing the following parameters:
molecular dynamics program: Materials Science Suites, Desmond module;
force field applied to calculations: Optimized Potentials for Liquid Simulations (OPLS)3e; and
simulation condition: NPT simulation, 50 ns, 300 K, 1 atm.

According to one or more embodiments, a method of forming patterns includes:
coating a metal-containing photoresist composition on a substrate; performing a heat treatment to form a photoresist film on the substrate (by drying and heating), exposing the metal-containing photoresist film; and developing the exposed film utilizing the developer composition for the metal-containing photoresist.

The developer composition for the metal-containing photoresist according to one or more embodiments has improved or optimized elements to maximize or increase the separation of a metal compound included in a photoresist through simulation. By applying a developer composition that meets these criterial, improved or enhanced solubility in an unexposed portion may be achieved (realized). For example, the developer composition may be enhanced to promote selective solubility of the unexposed portion of a metal-containing photoresist by modulating intermolecular interactions at the molecular level. This enhancement may be guided by molecular dynamics simulations, which quantify the hydrogen bonding behavior among the metal compound, organic solvent, and additive. By ensuring that the number of hydrogen bonds between the metal compound and the organic solvent, relative to those between the metal compound and the additive, falls within a defined range (greater than 0 and less than or equal to about 10), and that the number of hydrogen bonds between the additive and the organic solvent, relative to those between the metal compound and the additive, is greater than 0 and less than or equal to about 5, the developer composition may achieve a favorable interaction profile.

These hydrogen bonding parameters are not arbitrary; they are derived from simulations conducted utilizing the Desmond module of the Materials Science Suite, applying the OPLS3e force field under NPT conditions (50 ns, 300 K, 1 atm). The simulation results provide an average hydrogen bond count per frame, which serves as a predictive metric for solubility behavior in the unexposed region of the photoresist.

By applying a developer composition that satisfies these criteria, the method enables enhanced separation of the metal compound from the unexposed photoresist matrix. This results in improved or enhanced solubility of the unexposed portion, leading to higher development contrast, reduced line edge roughness (LER), and improved or enhanced critical dimension (CD) control-key performance metrics in advanced lithographic processes.

### BRIEF DESCRIPTION OF THE DRAWINGS

The accompanying drawings, together with the specification, illustrate embodiments of the subject matter of the present disclosure, and, together with the description, serve to explain principles of embodiments of the subject matter of the present disclosure.

FIGS. 1A-1C are cross-sectional views illustrating a process sequence in order to describe a method of forming patterns.

FIG. 2 is a schematic view illustrating the distribution of the entire composition of a metal compound, an organic solvent, and an additive in a metal-containing photoresist in an unexposed portion implemented for molecular dynamics calculations according to one or more embodiments of the present disclosure.

FIG. 3 is a schematic view illustrating the distribution of a composition that has reached an equilibrium state by performing molecular dynamics simulation on the composition according to FIG. 2.

### DETAILED DESCRIPTION

Hereinafter, the subject matter of the present disclosure will be described in more detail with reference to the accompanying drawings. It should be understood that certain functions, structures, or processes that are well known to those of ordinary skill in the art may not be provided or simplified in the following description in order to more clearly illustrate the features of the present disclosure.

The utilization of “may” if (e.g., when) describing embodiments of the present disclosure refers to “one or more embodiments of the present disclosure.”

As used herein, the singular forms “a,” “an,” and “the” are intended to include the plural forms as well, unless the context clearly indicates otherwise. The singular expression includes the plural expression unless the context clearly dictates otherwise.

As used herein, the term “and/or” or “or” includes any and all combinations of one or more of the associated listed items.

Throughout the present disclosure, the expressions, such as “at least one of,” “one of,” and “selected from,” if (e.g., when) preceding a list of elements, modify the entire list of elements and do not modify the individual elements of the list. For example, “at least one of a, b, or c,” “at least one selected from among a, b, and c,” “at least one selected from among a to c,” and/or the like indicates only a, only b, only c, both (e.g., simultaneously) a and b, both (e.g., simultaneously) a and c, both (e.g., simultaneously) b and c, all of a, b, and c, or variations thereof.

In the present disclosure, it will be understood that the term “comprise(s)/comprising,” “include(s)/including,” or “have/has/having” specifies the presence of stated features, integers, steps (e.g., acts or tasks), operations, elements, and/or components, but do not preclude the presence or addition of one or more other features, integers, steps (e.g., acts or tasks), operations, elements, components, and/or groups thereof. Also, the terms “comprise(s)/comprising,” “include(s)/including,” “have/has/having” or similar terms include or support the terms “consisting of” and “consisting essentially of,” indicating the presence of stated features, integers, steps (e.g., acts or tasks), operations, elements, and/or components, without or essentially without the presence of other features, integers, steps (e.g., acts or tasks), operations, elements, components, and/or groups thereof.

In the context of the present application and unless otherwise defined, the terms “use,” “using,” and “used” may be considered synonymous with the terms “utilize,” “utilizing,” and “utilized,” respectively.

As utilized herein, the terms “substantially,” “about,” or similar terms are used as terms of approximation and not as terms of degree and are intended to account for the inherent deviations in measured or calculated values that would be recognized by those of ordinary skill in the art. “About” as used herein is inclusive of the stated value and refers to as being within an acceptable range of deviation for the particular value as determined by one of ordinary skill in the art, considering the measurement in question and the error associated with measurement of the particular quantity (e.g., the limitations of the measurement system). For example, “about” may refer to as being within one or more standard deviations or within ±30%, ±20%, ±10%, or ±5% of the stated value. Also, it should be understood that, even if (e.g., when) the terms “about,” “approximately,” or “substantially” are not expressly recited in a given element (e.g., a claim element), the scope of such element is intended to include variations that are insubstantial or within the understanding of one of ordinary skill in the art. For example, numerical values and ranges provided herein are intended to include tolerances and measurement uncertainties that would be recognized by those skilled in the art, and the elements (e.g., claim elements) should be construed accordingly to encompass such equivalents.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, for example, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in the present disclosure is intended to include all higher numerical limitations subsumed therein. Accordingly, Applicant reserves the right to amend the disclosure, including the claims, to expressly recite any sub-range subsumed within the ranges expressly recited herein.

In order to clearly illustrate the present disclosure, the description and relationships may not be provided, and throughout the disclosure, substantially the same or similar configuration or arrangement elements may be designated by the same reference numerals. Also, because the size and thickness of each configuration or arrangement illustrated in the drawing are arbitrarily shown for better understanding and ease of description, embodiments of the present disclosure are not necessarily limited thereto.

In the drawings, the thickness of layers, films, panels, regions, and/or the like may be exaggerated for clarity. In the drawings, the thickness of a part of layers, regions, and/or the like may be exaggerated for better understanding and ease of description.

It will be understood that if (e.g., when) an element, such as a layer, a film, a region, or a substrate, is referred to as being “on” or “above” another element, it may be directly on or directly above the other element or intervening elements may also be present therebetween. In contrast, if (e.g., when) an element is referred to as being “directly on” or “directly above” another element, there are no intervening elements present therebetween.

Hereinafter, a developer composition for a metal-containing photoresist according to one or more embodiments is described.

The developer composition for a metal-containing photoresist according to one or more embodiments may be a developer composition applied to a metal-containing photoresist having an exposed portion and an unexposed portion, and
includes a metal compound, an organic solvent, and an additive,
in the unexposed portion,
the number of hydrogen bonds between the metal compound and the organic solvent relative to the number of hydrogen bonds between the metal compound and the additive in the metal-containing photoresist may be greater than 0 and less than or equal to about 10, and
the number of hydrogen bonds between the additive and the organic solvent relative to the number of hydrogen bonds between the metal compound and the additive in the metal-containing photoresist may be greater than 0 and less than or equal to about 5.

The number of hydrogen bonds may be calculated by counting each frame after molecular dynamics simulation and taking an average value of the each frame:

Molecular dynamics program: Materials Science Suites, Desmond module,

Force field applied to calculations: OPLS3e (OPLS3e refers to a version of the Optimized Potentials for Liquid Simulations (OPLS) force field. It may be used in molecular dynamics (MD) simulations to model the behavior of molecules by calculating the potential energy of a system based on atomic interactions), and

Simulation condition: NPT simulation, 50 ns, 300 K, 1 atm (NPT simulation refers to a type (kind) of molecular dynamics (MD) simulation that maintains constant: N: Number of particles (atoms or molecules); P: Pressure; and T: Temperature. This is also known as the isothermal-isobaric ensemble).

This hydrogen bonding analysis provides a molecular-level understanding of how the developer composition interacts with the photoresist components, for example, in the unexposed regions. By quantifying the relative strengths and frequencies of hydrogen bonds among the metal compound, organic solvent, and additive, the formulation can be fine-tuned to achieve selective solubility. A higher number of hydrogen bonds between the metal compound and the organic solvent, compared to those with the additive, promotes dissolution of the unexposed portion without adversely affecting the exposed regions. This selective interaction is critical for achieving high-resolution pattern development, minimizing or reducing line edge roughness (LER), and ensuring uniform critical dimensions (CD) in advanced lithographic processes.

For example, the number of hydrogen bonds between the metal compound and the organic solvent relative to the number of hydrogen bonds between the metal compound and the additive in the metal-containing photoresist may be greater than 0 and less than or equal to about 8, for example, greater than 0 and less than or equal to about 6, and, for example, greater than 0 and less than or equal to about 5.

For example, the number of hydrogen bonds between the metal compound and the organic solvent relative to the number of hydrogen bonds between the metal compound and the additive in the metal-containing photoresist may be greater than 0 and less than or equal to about 4, for example, greater than or equal to about 0.2 and less than or equal to about 4.

For example, the number of hydrogen bonds between the additive and the organic solvent relative to the number of hydrogen bonds between the metal compound and the additive in the metal-containing photoresist may be greater than 0 and less than or equal to about 4.

The number of molecules of the metal compound having a maximum intermolecular distance of the metal compound of less than or equal to about 3.5 Å may be greater than 0 and less than or equal to 10.

For example, the number of molecules of the metal compound having a maximum intermolecular distance of the metal compound of less than or equal to about 3.5 Å may be about 1 to about 10 or less, for example, about 2 to about 10 or less, for example, about 3 to about 10 or less, and, for example, about 4 to about 10 or less.

For example, the number of molecules of the metal compound having a maximum intermolecular distance of the metal compound of less than or equal to about 3.5 Å may be about 5 to about 10.

The number of molecules may be calculated by counting each frame after molecular dynamics simulation and taking an average value of the each frame.

For example, the metal compound may include at least one selected from among tin (Sn), tellurium (Te), and antimony (Sb).

For example, the metal compound may include Sn.

Examples of an organic solvent included in a developer composition for a metal-containing photoresist according to one or more embodiments may include at least one selected from among an ether, an alcohol, a glycol ether, an aromatic hydrocarbon compound, a ketone, and an ester, but embodiments of the present disclosure are not limited thereto. For example, the organic solvent may include ethylene glycol monomethyl ether, ethylene glycol monoethyl ether, methyl cellosolve acetate, ethyl cellosolve acetate, diethylene glycol methyl ether, diethylene glycol ethyl ether, propylene glycol, propylene glycol methyl ether (PGME), propylene glycol methyl ether acetate (PGMEA), propylene glycol ethyl ether, propylene glycol ethyl ether acetate, propylene glycol propyl ether acetate, propylene glycol butyl ether, propylene glycol butyl ether acetate, ethanol, propanol, isopropyl alcohol, isobutyl alcohol, 4-methyl-2-pentanol (or referred to as methyl isobutyl carbinol (MIBC)), hexanol, 1-methoxy-2-propanol, 1-ethoxy-2-propanol, ethylene glycol, propylene glycol, heptanone, propylene carbonate, butylene carbonate, toluene, xylene, methylethylketone, cyclopentanone, cyclohexanone, 2-hydroxy ethyl propionate, 2-hydroxy-2-methyl ethyl propionate, ethoxy ethyl acetate, hydroxy ethyl acetate, 2-hydroxy-3-methylmethyl butanoate, 3-methoxy methyl propionate, 3-methoxy ethyl propionate, 3-ethoxy ethyl propionate, 3-ethoxy methyl propionate, methyl pyruvate, ethyl pyruvate, ethyl acetate, butyl acetate, ethyl lactate, butyl lactate, gamma-butyrolactone, methyl-2-hydroxyisobutyrate, methoxybenzene, n-butyl acetate, 1-methoxy-2-propyl acetate, methyl methoxy propionate, ethyl ethoxy propionate, or a combination thereof, but embodiments of the present disclosure are not limited thereto.

Examples of the additive included in the developer composition for the metal-containing photoresist according to one or more embodiments may include an organic acid, phosphoric acid, phosphorous acid, a diol compound, and/or a diketone compound, but embodiments of the present disclosure are not limited thereto.

The developer composition for a metal-containing photoresist according to one or more embodiments may further include at least one other additive selected from among a surfactant, a dispersant, a hygroscopic agent, and a coupling agent.

In one or more embodiments, a method of forming patterns may include a developing step (e.g., act or task) utilizing the developer composition for a metal-containing photoresist as described in one or more embodiments. For example, the manufactured pattern may be a negative-tone (type/kind) photoresist pattern.

A method for forming patterns according to one or more embodiments may include coating a metal-containing photoresist composition on a substrate, performing heat treatment in which a metal-containing photoresist film is formed on the substrate by drying and heating, exposing the metal-containing photoresist film, and developing utilizing the developer composition for the metal-containing photoresist. For example, the method may begin by applying a uniform layer of a metal-containing photoresist composition onto a substrate utilizing techniques such as spin coating, slit coating, and/or inkjet printing. This may be followed by a soft bake or pre-bake step (e.g., act or task), during which the coated substrate is heated to remove residual solvent and promote adhesion of the photoresist film to the substrate surface. After the film is stabilized (formed), it may be exposed to a radiation source, such as EUV, e-beam, or deep ultraviolet (DUV) light, through a photomask or direct-write system to define the desired pattern. The exposed regions may undergo a chemical transformation, such as crosslinking, that alters their solubility. The substrate may be then subjected to a development step (e.g., act or task) utilizing the developer composition as described in one or more embodiments, which selectively dissolves the unexposed regions of the photoresist, thereby revealing the patterned features with high fidelity.

For example, the forming of the patterns utilizing the metal-containing photoresist composition may include coating a metal-containing photoresist composition on a substrate on which a thin film is formed by spin coating, slit coating, inkjet printing, and/or the like, and drying the coated metal-containing photoresist composition to forma resist layer. The metal-containing photoresist composition may include a tin-based compound, and, for example, the tin-based compound may include at least one selected from among an alkyl tin oxo group, an alkyl tin carboxyl group, and an alkyl tin hydroxyl group.

Subsequently, a first heat treatment process may be performed to heat the substrate on which the metal-containing photoresist film is formed. The first heat treatment process may be performed at a temperature of about 80° C. to about 120° C.

In this process, the solvent may be evaporated and the metal-containing photoresist film may be more firmly or suitably adhered to the substrate. For example, this first heat treatment may also be referred to as a soft bake or pre-bake and may serve multiple functions in the patterning process. It may facilitate the controlled evaporation of residual solvents from the coated photoresist layer, which is for achieving uniform (e.g., substantially uniform) film thickness and preventing/reducing defects, such as bubbling and/or delamination, during subsequent processing. Also, this thermal step (e.g., act or task) may promote partial densification of the metal-containing photoresist, enhancing its mechanical stability and adhesion to the underlying substrate. The selected temperature range of about 80° C. to about 120° C. may be to ensure sufficient or suitable solvent removal without initiating premature chemical reactions or degradation of the photoresist components. This step (e.g., act or task) may be for ensuring consistent exposure and development performance in later stages of the lithographic process.

Then, the photoresist film may be selectively exposed.

Examples of light that may be utilized in the exposure process may include not only light having relatively low energy wavelengths, such as i-line (wavelength of 365 nm), KrF excimer laser (wavelength of 248 nm), and/or ArF excimer laser (wavelength of 193 nm), but also light having relatively high energy wavelengths, such as extreme ultraviolet (EUV; wavelength of 13.5 nm) and/or the like, and other sources, such as electron beam (e-beam) and/or the like.

For example, the light for exposure according to one or more embodiments may be light having a wavelength range of about 5 nm to about 150 nm, such as extreme ultraviolet (EUV; wavelength of 13.5 nm) and/or the like, and other sources such as, electron beam (e-beam).

In the step (e.g., act or task) of forming the photoresist pattern, a negative-tone (type/kind) pattern may be formed.

The exposed portion of the photoresist film may form a polymer through a crosslinking reaction, such as condensation between organometallic compounds, and thus may have a different solubility from the unexposed portion of the photoresist film.

Then, a second heat treatment process may be performed on the substrate. The second heat treatment process may be performed at a temperature of about 90° C. to about 200° C. For example, the second heat treatment may also be referred to as a post-exposure bake (PEB) and may be a step (e.g., act or task) that follows the exposure of the metal-containing photoresist film. This thermal process may be conducted at a temperature ranging from about 90° C. to about 200° C., depending on the specific formulation of the photoresist and the desired patterning outcome. The primary function of the PEB may be to promote chemical reactions, such as crosslinking and/or condensation, within the exposed regions of the photoresist. These reactions may enhance the structural integrity and reduce the solubility of the exposed areas, thereby enabling the formation of a negative-tone pattern during development. The elevated temperature may accelerate the mobility of reactive species and facilitates the formation of a robust polymer network, for example, in metal-organic systems like tin-based photoresists. Proper control of the PEB conditions may be for achieving high-resolution features, minimizing/reducing line edge roughness (LER), and ensuring consistent critical dimension (CD) control across the substrate.

By performing the second heat treatment process, the exposed portion of the photoresist film may become difficult to be dissolved in a developer.

For example, the photoresist pattern corresponding to the negative-tone (type/kind) tone image may be completed by dissolving and then removing the photoresist film corresponding to the unexposed portion utilizing the photoresist developer as described in one or more embodiments.

As described in one or more embodiments, the photoresist pattern formed by exposure to not only light having relatively low energy wavelengths, such as i-line (wavelength of 365 nm), KrF excimer laser (wavelength of 248 nm), and/or ArF excimer laser (wavelength of 193 nm), but also light having relatively high energy wavelengths, such as extreme ultraviolet (EUV; wavelength of 13.5 nm) and/or the like, and other sources having high energy, such as an electron beam (e-beam), may have a thickness width of about 5 nm to about 100 nm. For example, the photoresist pattern may be formed to have a thickness width of about 5 nm to about 90 nm, about 5 nm to about 80 nm, about 5 nm to about 70 nm, about 5 nm to about 60 nm, about 5 nm to about 50 nm, about 5 nm to about 40 nm, about 5 nm to about 30 nm, or about 5 nm to about 20 nm.

In one or more embodiments, the photoresist pattern may have a pitch having a half-pitch of less than or equal to about 50 nm, for example, less than or equal to about 40 nm, for example, less than or equal to about 30 nm, for example, less than or equal to about 20 nm, for example, less than or equal to about 15 nm, and a line width roughness of less than or equal to about 10 nm, less than or equal to about 5 nm, less than or equal to about 3 nm, or less than or equal to about 2 nm.

The method as described herein may leverage the tailored hydrogen bonding interactions of the developer composition to achieve high-resolution pattern formation in metal-containing photoresists. By selectively dissolving the unexposed regions while preserving the integrity of the exposed, crosslinked areas, the developer composition enables precise pattern transfer with minimal or reduce line edge roughness (LER) and excellent or suitable critical dimension (CD) control. This may be advantageous or beneficial for next-generation lithographic techniques, such as EUV and e-beam lithography, where sub-20 nm features and tight process windows are required or desired. The ability to form patterns with half-pitches below about 30 nm and LER values under about 3 nm demonstrates the effectiveness of the developer composition in supporting advanced semiconductor manufacturing nodes.

Hereinafter, a method of forming patterns is described in more detail with reference to the drawings.

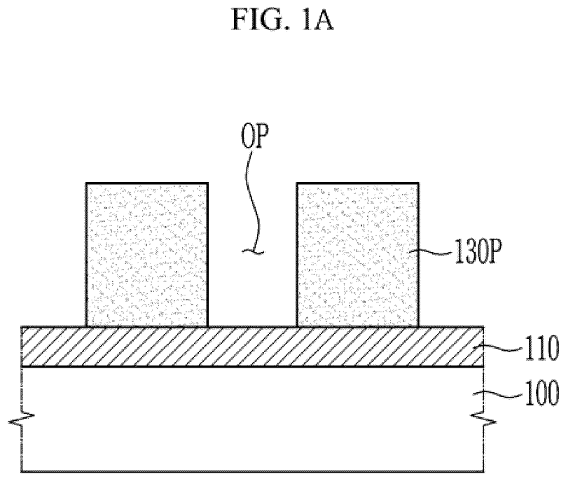

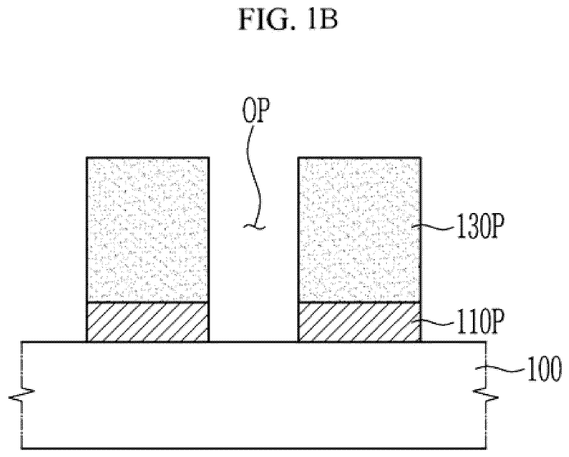

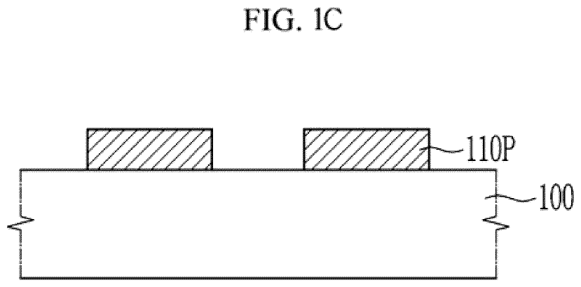

FIGS. 1A-1C are cross-sectional views illustrating a process sequence in order to describe a method of forming patterns.

Referring to FIG. 1A, the exposed photoresist film may be developed to form a photoresist pattern 130P.

In one or more embodiments, the exposed photoresist film may be developed to remove an unexposed portion of the photoresist film, and the photoresist pattern 130P including the exposed portion of the photoresist film may be formed. The photoresist pattern 130P may include a plurality of openings OP.

In one or more embodiments, the development of the photoresist film may be performed through a negative-tone development (NTD) process. Herein, the developer composition for the metal-containing photoresist according to one or more embodiments may be utilized as a developer composition.

Referring to FIG. 1B, the photoresist pattern 130P may be utilized to process a feature layer 110 in the result of FIG. 1A.

For example, the feature layer 110 may be processed through one or more suitable processes of etching a feature layer 110 exposed through the openings OP of the photoresist pattern 130P, injecting impurity ions into the feature layer 110, forming an additional film on the feature layer 110 through the openings OP, deforming a portion of the feature layer 110 through the openings OP, and/or the like. FIG. 1B illustrates an example process of processing a feature pattern 110P by etching the feature layer 110 exposed through the openings OP.

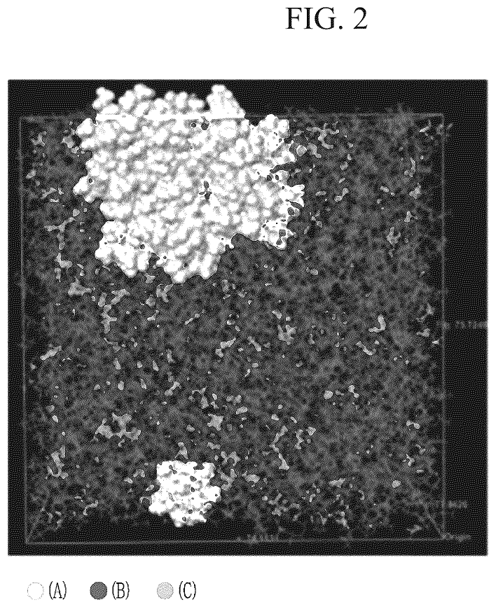

Referring to FIG. 1C, the photoresist pattern 130P remaining on the feature pattern 110P may be removed in the result of FIG. 2. In order to remove the photoresist pattern 130P, an ashing process and/or a stripping process may be used. The feature pattern 110P may be on a substrate 100.

Hereinafter, one or more embodiments of the present disclosure will be described in more detail through examples relating to the preparation of the developer composition for the metal-containing photoresist as described in one or more embodiments. However, embodiments of the present disclosure are not limited by the following examples.

The molecular dynamics simulation results data are shown in Table 1.

For molecular dynamics calculations, the Materials Science Suites program and Desmond module were used to produce initial compositions including organic solvents and additives, adjusting the number of additives and organic solvents according to additive conditions, so that (e.g., such that) the total number of molecules of the metal compound, the additive, and the organic solvent was 2000. Herein, the metal compound was designed to have a simulated structure implemented for a molecular dynamics simulation and consisting of a cluster of 21 Sn molecules (e.g., (t-Bu)₃Sn₃(O₂CH)₅(OH)₂O), which were trimers.

FIG. 2 is an improved or optimized state of the initial composition.

FIG. 2 is a schematic view illustrating the distribution of the entire composition of a metal compound, an organic solvent, and an additive in a metal-containing photoresist in an unexposed portion implemented for molecular dynamics calculations according to one or more embodiments of the present disclosure.

Referring to FIG. 2, in the initial composition of the unexposed portion, the metal compound (A) was not evenly (e.g., substantially evenly) distributed in the organic solvent (B) and the additive (C), which confirmed that no dissolution occurred by the developer.

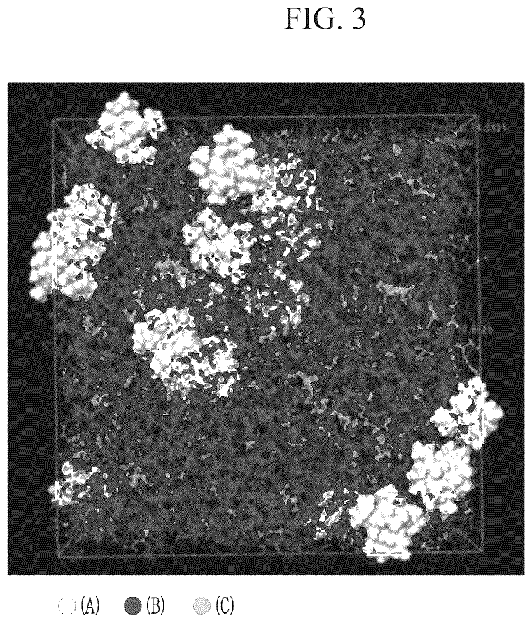

Subsequently, the equilibrium state calculation was performed by carrying out a molecular dynamic simulation in an NPT ensemble method (e.g., Thermostat method: Nose-Hoover chain, Barostat method: Martyna-Tobias-Klein) under conditions of a pressure of 1 atm and a temperature of 300 K for 50 ns. Herein, the distribution of the composition that reached the equilibrium state is shown in FIG. 3.

Referring to FIG. 3, the composition that reached the equilibrium state in the unexposed portion exhibited that the metal compound (A) was evenly (e.g., substantially evenly) distributed in the organic solvent (B) and the additive (C), which confirmed that dissolution sufficiently or suitably occurred by the developer.

In one or more embodiments, the number of hydrogen bonds was obtained by counting the number of hydrogen bonds between the metal compound and the organic solvent and the additive for each frame in the entire cell and then, calculating an average value of the frame values.

Also, the number of the metal compound was obtained by counting the number of molecules forming the metal compound within a distance of 3.5 Å for each frame after performing the molecular dynamics simulation by utilizing a cluster analysis module of Materials Science Suites program and then, calculating an average value of the frame values.

TABLE 1

| | | | | | The number of hydrogen bonds | | |
|---|---|---|---|---|---|---|---|
| | Unexposed | | | | | Metal | |
| | portion | | The number of | | Metal | compound | Additive |
| | | Organic | simulations | | compound | and | and |
| | Additive | solvent | | Organic | and | organic | organic |
| | (wt %) | (wt %) | Additive | solvent | additive | solvent | solvent |
| Example 1 | C1 | S1 | 35 | 1965 | 22.28 | 16.94 | 20.47 |
| | (1) | (99) | | | | | |
| Example 2 | C1 | S1 | 155 | 1845 | 62.11 | 10.97 | 175.49 |
| | (5) | (95) | | | | | |
| Example 3 | C2 | S1 | 25 | 1975 | 31.5 | 16.9 | 29.0 |
| | (1) | (99) | | | | | |
| Example 4 | C3 | S1 | 20 | 1980 | 20.2 | 17.7 | 25.7 |
| | (1) | (99) | | | | | |
| Example 5 | C4 | S2 | 130 | 1870 | 5.6 | 20.9 | 13.7 |
| | (5) | (95) | | | | | |
| Example 6 | C4 | S2 | 380 | 1620 | 15 | 16.6 | 33.2 |
| | (15) | (85) | | | | | |
| Example 7 | C4 | S2 | 498 | 1502 | 20.2 | 19.3 | 40.8 |
| | (20) | (80) | | | | | |
| Example 8 | C4 | S1 | 228 | 1772 | 13.7 | 18 | 12.5 |
| | (10) | (90) | | | | | |
| Example 9 | C5 | S1 | 150 | 1850 | 13.94 | 18.94 | 15.3 |
| | (10) | (90) | | | | | |
| Example 10 | C6 | S1 | 13 | 1987 | 14.43 | 17.43 | 10.12 |
| | (0.5) | (95.5) | | | | | |
| Example 11 | C7 | S1 | 185 | 1815 | 16.8 | 14.1 | 61.5 |
| | (10) | (90) | | | | | |
| Comparative | C1 | S1 | 300 | 1700 | 68.06 | 9.08 | 350 |
| Example 1 | (10) | (90) | | | | | |
| Comparative | C4 | S2 | 26 | 1974 | 1.51 | 20.08 | 2.65 |
| Example 2 | (1) | (99) | | | | | |
| Comparative | C4 | S3 | 200 | 1800 | 4.16 | 73.23 | 174.43 |
| Example 3 | (10) | (90) | | | | | |
| Comparative | C8 | S3 | 20 | 1980 | 5.44 | 78.59 | 95.35 |
| Example 4 | (1) | (99) | | | | | |
| Comparative | C7 | S2 | 210 | 1790 | 11 | 17.8 | 90.1 |
| Example 5 | (10) | (90) | | | | | |
| Comparative | C9 | S2 | 430 | 1570 | 12.9 | 16.2 | 196.4 |
| Example 6 | (20) | (80) | | | | | |
| Comparative | C10 | S1 | 420 | 1580 | 67.3 | 14.3 | 425.4 |
| Example 7 | (20) | (80) | | | | | |

### Additive

C1: Propionic acid
C2: Succinic acid
C3: Fumaric acid
C4: Acetyl acetone
C5: Trifluoroacetylacetone
C6: Methylphosphonic acid
C7: Maltol
C8: Phosphorous acid
C9: Tropolone
C10: Catechol

### Organic Solvent

S1: n-butyl acetate
S2: Propylene glycol methyl ether acetate (PGMEA)
S3: Methyl isobutyl carbinol (MIBC)

### Evaluation: Defect Evaluation

An organometallic compound having the structure of Chemical Formula C was dissolved in 4-methyl-2-pentanol at a concentration of 1 wt %, and then filtered through a 0.1 μm polytetrafluoroethylene (PTFE) syringe filter to prepare a photoresist composition.

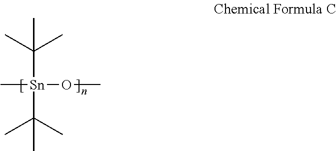

The prepared organic metal-containing photoresist (PR) composition was spin-coated on an 8-inch wafer at 1,500 rpm for 30 seconds, and then heat-treated at 160° C. for 60 seconds to produce a coated wafer.

The developer compositions according to Examples 1 to 11 and Comparative Examples 1 to 7 were respectively applied to proceed with a development process at a spin speed of 1,500 rpm for 30 seconds and then, cured at 240° C. for 60 seconds.

After completing the curing process, wafer defects were measured by utilizing a surface inspection device (e.g., SurfScan SP2, KLA Tencor Corp.)

With reference to the number of defect with a size of less than or equal to 0.3 μm, if the number of defect was less than 150, ‘Good’ was given, but if the number of defects was greater than or equal to 150, ‘Bad’ was given.

TABLE 2

| | Hydrogen bond ratio (Normalized) | | | | |
|---|---|---|---|---|---|
| | | Metal | | | |
| | Metal | compound | Additive | Metal | |
| | compound | and | and | compound | |
| | and | organic | organic | The number | Defect |
| | additive | solvent | solvent | of molecules | SP2 |
| Example 1 | 1.0 | 0.8 | 0.9 | 5.49 | Good |
| Example 2 | 1.0 | 0.2 | 2.8 | 5.02 | Good |
| Example 3 | 1.0 | 0.5 | 0.9 | 6.10 | Good |
| Example 4 | 1.0 | 0.9 | 1.3 | 6.64 | Good |
| Example 5 | 1.0 | 3.7 | 2.4 | 8.71 | Good |
| Example 6 | 1.0 | 1.1 | 2.2 | 9.67 | Good |
| Example 7 | 1.0 | 1.0 | 2.0 | 7.65 | Good |
| Example 8 | 1.0 | 1.3 | 0.9 | 5.32 | Good |
| Example 9 | 1.0 | 1.4 | 1.1 | 6.24 | Good |
| Example 10 | 1.0 | 1.2 | 0.7 | 7.07 | Good |
| Example 11 | 1.0 | 0.8 | 3.7 | 7.11 | Good |
| Comparative | 1.0 | 0.1 | 5.1 | 4.86 | Bad |
| Example 1 | | | | | |
| Comparative | 1.0 | 13.3 | 1.8 | 8.63 | Bad |
| Example 2 | | | | | |
| Comparative | 1.0 | 17.6 | 41.9 | 8.21 | Bad |
| Example 3 | | | | | |
| Comparative | 1.0 | 14.4 | 17.5 | 14.86 | Bad |
| Example 4 | | | | | |
| Comparative | 1.0 | 1.6 | 8.2 | 10.80 | Bad |
| Example 5 | | | | | |
| Comparative | 1.0 | 1.3 | 15.2 | 13.30 | Bad |
| Example 6 | | | | | |
| Comparative | 1.0 | 0.2 | 6.3 | 6.33 | Bad |
| Example 7 | | | | | |

Referring to Table 2, when the developer compositions for the metal-containing photoresist according to Examples 1 to 11 are applied, excellent or suitable dissolution characteristics are exhibited in the unexposed portions compared to when the developer compositions for the metal-containing photoresist according to Comparative Examples 1 to 7 are applied.

A patterning device, a developer composition manufacturing device, and/or any other relevant devices or components according to one or more embodiments of the present invention described herein may be implemented utilizing any suitable hardware, firmware (e.g., an application-specific integrated circuit), software, or a combination of software, firmware, and hardware. For example, the one or more suitable components of the device may be formed on one integrated circuit (IC) chip or on separate IC chips. Further, the one or more suitable components of the device may be implemented on a flexible printed circuit film, a tape carrier package (TCP), a printed circuit board (PCB), or formed on one substrate. Further, the one or more suitable components of the device may be a process or thread, running on one or more processors, in one or more computing devices, executing computer program instructions and interacting with other system components for performing the one or more suitable functionalities described herein. The computer program instructions may be stored in a memory which may be implemented in a computing device using a standard memory device, such as, for example, a random access memory (RAM). The computer program instructions may also be stored in other non-transitory computer readable media such as, for example, a CD-ROM, flash drive, and/or the like. Also, a person of skill in the art should recognize that the functionality of one or more suitable computing devices may be combined or integrated into a single computing device, or the functionality of a particular computing device may be distributed across one or more other computing devices without departing from the scope of the present disclosure.

Hereinbefore, certain embodiments of the present disclosure have been described and illustrated, however, it should be apparent to a person having ordinary skill in the art that the present disclosure is not limited to the embodiments as described and may be suitably modified and transformed without departing from the spirit and scope of the present disclosure. Accordingly, the modified or transformed embodiments as such may not be understood separately from the technical ideas and aspects of one or more embodiments of the present disclosure, and the modified embodiments may be within the scope of the appended claims and equivalents thereof.

### REFERENCE NUMERALS

100: substrate OP: opening
110: feature layer 110P: feature pattern
130P: photoresist pattern

What is claimed is:

1. A developer composition, comprising:
a metal compound;
an organic solvent; and
an additive;
wherein the developer composition is to be applied to a metal-containing photoresist having an exposed portion and an unexposed portion, and
wherein, in the unexposed portion:
a ratio of the number of hydrogen bonds between the metal compound and the organic solvent to the number of hydrogen bonds between the metal compound and the additive is greater than 0 and less than or equal to 10; and
a ratio of the number of hydrogen bonds between the additive and the organic solvent to the number of hydrogen bonds between the metal compound and the additive is greater than 0 and less than or equal to 5,
wherein the number of hydrogen bonds is determined by averaging the number of hydrogen bonds per frame over the course of a molecular dynamics simulation; and
wherein the developer composition is a developer composition for the metal-containing photoresist.

2. The developer composition as claimed in claim 1, wherein the ratio of the number of hydrogen bonds between the metal compound and the organic solvent to the number of hydrogen bonds between the metal compound and the additive in the metal-containing photoresist is greater than 0 and less than or equal to 4.

3. The developer composition as claimed in claim 1, wherein the ratio of the number of hydrogen bonds between the metal compound and the organic solvent to the number of hydrogen bonds between the metal compound and the additive in the metal-containing photoresist is in the range of 0.2 to 4.

4. The developer composition as claimed in claim 1, wherein the ratio of the number of hydrogen bonds between the additive and the organic solvent to the number of hydrogen bonds between the metal compound and the additive in the metal-containing photoresist is greater than 0 and less than or equal to 5.

5. The developer composition as claimed in claim 1,
wherein a number of molecules of the metal compound having a maximum intermolecular distance of less than or equal to 3.5 Å is greater than 0 and less than or equal to 10, and
wherein the number of such molecules is determined by averaging the count per frame over the course of the molecular dynamics simulation.

6. The developer composition as claimed in claim 1, wherein a number of molecules of the metal compound having a maximum intermolecular distance of less than or equal to 3.5 Å is greater than or equal to 5 and less than or equal to 10.

7. The developer composition as claimed in claim 1, wherein the metal compound comprises at least one metal selected from among Sn, Te, Sb, and combinations thereof.

8. The developer composition as claimed in claim 1, wherein the metal compound comprises Sn.

9. The developer composition as claimed in claim 1, wherein the organic solvent comprises at least one selected from among an ether, an alcohol, a glycol ether, an aromatic hydrocarbon compound, a ketone, an ester, and combinations thereof.

10. The developer composition as claimed in claim 1, wherein the organic solvent comprises at least one selected from among n-butyl acetate, propylene glycol methyl ether acetate (PGMEA), methyl isobutyl carbinol (MIBC), and combinations thereof.

11. The developer composition as claimed in claim 1, wherein the additive comprises at least one selected from among an organic acid, phosphoric acid, phosphorous acid, a diol compound, a diketone compound, and combinations thereof.

12. The developer composition as claimed in claim 11, wherein the additive comprises at least one selected from among propionic acid, succinic acid, fumaric acid, acetyl acetone, trifluoroacetylacetone, methylphosphonic acid, maltol, phosphorous acid, tropolone, catechol, and combinations thereof.

13. The developer composition as claimed in claim 1, wherein the additive further comprises at least one other additive selected from among a surfactant, a dispersant, a hygroscopic agent, a coupling agent, and combinations thereof.

14. The developer composition as claimed in claim 1, wherein the number of hydrogen bonds is determined based on the molecular dynamics simulation performed with:
a molecular dynamics program comprising the Desmond module of the Materials Science Suite;
a force field comprising OPLS3e; and
simulation conditions comprising an NPT ensemble, a simulation time of 50 nanoseconds, a temperature of 300 K, and a pressure of 1 atm.

15. A method comprising:
coating a metal-containing photoresist composition on a substrate;
performing a heat treatment to form a metal-containing photoresist film on the substrate by drying and heating;
exposing the metal-containing photoresist film; and
developing the exposed film utilizing the developer composition as claimed in claim 1,
where the method is a method of forming patterns.

16. The method as claimed in claim 15, further comprising performing a second heat treatment after the exposing of the metal-containing photoresist film and before the developing of the exposed film utilizing the developer composition, wherein the second heat treatment is performed at a temperature in the range of 90° C. to 200° C.

17. The method as claimed in claim 15, wherein:
the performing of the heat treatment is performed at a temperature of 80° C. to 120° C.

18. The method as claimed in claim 15, wherein the metal-containing photoresist composition comprises at least one tin-based compound selected from the group consisting of an alkyl tin oxo group, an alkyl tin carboxyl group, an alkyl tin hydroxyl group, and combinations thereof.

19. The method as claimed in claim 15, wherein the pattern formed has a half-pitch of less than or equal to 20 nm and a line edge roughness of less than or equal to 3 nm.

20. The method as claimed in claim 15, wherein the number of hydrogen bonds utilized to evaluate the developer composition is determined based on molecular dynamics simulation performed with:
a molecular dynamics program comprising the Desmond module of the Materials Science Suite;
a force field comprising OPLS3e; and
simulation conditions comprising an NPT ensemble, a simulation time of 50 nanoseconds, a temperature of 300 K, and a pressure of 1 atm.
